(19) United States
(12) Patent Application Publication
(10) Pub. No.: US 2005/0197565 A1
(43) Pub. Date: Sep. 8, 2005

(54) Audio communication apparatus for MRI apparatus

(75) Inventors: Kazuo Yagi, Arakawa-ku (JP); Kenji Muto, Sumida-ku (JP); Kaoru Terasaki, Mitaka-shi (JP); Takeo Nishino, Mitaka-shi (JP); Toshiyuki Hirata, Mitaka-shi (JP)

Correspondence Address: OLIFF & BERRIDGE, PLC, P.O. BOX 19928, ALEXANDRIA, VA, 22320, US

(73) Assignee: AZDEN CORPORATION, Mitaka-shi (JP)

(21) Appl. No.: 10/948,280

(22) Filed: Sep. 24, 2004

(30) Foreign Application Priority Data
Mar. 2, 2004 (JP) 2004-057609

(51) Int. Cl. A61B 5/05

(52) U.S. Cl. 600/418

(57) ABSTRACT

An audio communication apparatus for an MRI apparatus performs unidirectional or bidirectional audio communication between a subject in a gantry provided in an examination room and an operator in a control room. An acoustic transducer for transmission and/or reception provided on the subject side is formed by a bone conduction microphone and/or speaker. The bone conduction microphone/speaker transmits a sound to or from the subject by contacting the subject directly or via a protection member, without relying on air as a transmission medium. The apparatus carries out audio transmission without being affected by noise, even in a high-noise environment. The apparatus allows the use of earplugs or sound insulation earmuffs.

### INCORPORATION BY REFERENCE

The disclosure of Japanese Patent Application No. 2004-057609 filed on Mar. 2, 2004 including the specification, drawings and abstract is incorporated herein by reference in its entirety.

### BACKGROUND OF THE INVENTION

1. Field of the Invention

The present invention relates to an audio communication apparatus that performs unidirectional or bidirectional communication between the side of a subject in an MRI gantry (image taking system) installed in an examination room and the side of an operation console installed in a control room in an MRI apparatus that is installed in hospitals, other medical care facilities, etc.

2. Description of the Related Art

MRI apparatuses acquire images for diagnostics and treatments in medical care by utilizing the nuclear magnetic resonance (NMR) phenomenon. Since the MRI apparatuses provide high-contrast images of organs, tissues, etc., inside a human body, the MRI apparatuses play an important role in diagnostics of cancers, tumors, and other diseases. Since the MRI apparatuses have features of being noninvasive to human bodies, and involving no radiation exposure unlike X-ray CT apparatuses, etc., the dead for MRI apparatuses is further increasing these days.

However, although the MRI apparatuses are very effective as a medical imaging diagnostic apparatus, the MRI apparatuses have the following problems to subject persons.

The MRI gantry space that an MRI apparatus has as an examination space for housing a subject person is designed with a very small examination area During operation of the gantry (during the MRI examination), loud noise as high as or higher than 100 dB are produced. Still further, the time required for the examination is inconveniently long, ranging from several tens of minutes to more than one hour.

During the MRI examination, a subject is exposed to considerably loud noise while the subject is placed within a closed small examination space (examination room) where the available means for communication with an external device is considerably poor. Therefore, the mental and physical burden of the subject is considerable. Thus, the MRI apparatus causes the subject person to feel loneliness and a great deal of discomfort. In addition, the MRI apparatus is likely to cause temporary deafness. As a result, countermeasures for the noise are extremely important.

One countermeasure against the aforementioned noise is a method in which the entire MRI apparatus is constructed so as to reduce noise. However, this method is complicated, and has many problems that are difficult to solve, so, at present, the method is regarded merely as a future solution. A most familiar and simplest countermeasure against noise is a method in which ears are blocked with earplugs or sound insulation earmuffs.

However, a problem of the method that uses earplugs or earmuffs to block sounds is that the earplugs or earmuffs also block out necessary sounds and voices, for example, voices from an operator of the MRI apparatus.

In a related-art MRI apparatus technology disclosed in Japanese Patent Application Laid-Open Publication No. 2003-126058, an apparatus for communication between a subject and an operator is provided with a noise countermeasure. Specifically, in this communication apparatus, voices and sounds are transmitted directly into the earholes of a subject via sound transmission tubes while the ears are blocked by ear-housing parts.

In this related-art apparatus, sounds (including voices) are transmitted via air, and noise sometimes mix in via a subject as earhole portion. Thus, this cannot be considered to be an effective audio transmission method in a noisy environment where the noise level reaches 100 dB or higher. In the present situation, therefore, a great ability of the human auditory sense to exclude noise (ability to selectively hear necessary voices and sounds despite a noisy background) is still a most widely relied-upon noise countermeasure.

### SUMMARY OF THE INVENTION

The present invention has been accomplished in view of the aforementioned circumstances. It is a first object of the present invention to provide an audio communication apparatus for an MRI apparatus which makes it possible to clearly hear the sounds and voices transmitted from the operation console side to the subject side without relying on the great ability of the human auditory sense to exclude noise, even in a high-noise environment during operation of the HI apparatus, and also makes it possible to always clearly hear sounds and voices at the operation console side if the sounds and voices are transmitted from the subject side to the operation console side.

It is a second object of the present invention to prevent the noise occurrence in MRI images and the distortion and depletion of MRI image that are ascribable to direct audio transmission type acoustic transducers formed of a magnetic material such as iron or the like.

A third object of the present invention is to realize the aforementioned direct audio transmission type acoustic transducer in a specific fashion.

A fourth object of the present invention is to provide an audio communication apparatus for an MRI apparatus which is easy for a subject to use and which makes it possible to more clearly hear the sounds and voices from the operation console side without the need to separately prepare earplugs or sound insulation earmuffs.

A fifth object of the present invention is to provide an audio communication apparatus for an MRI apparatus which makes it possible to clearly hear the sounds and voices transmitted from the operation console side to the subject side without relying on the great ability of the human auditory sense to exclude noise, even mi a high-noise environment during operation of the MRI apparatus, and which is able to alleviate the mental burden of the subject before, during and after the MRI examination

In order to achieve the aforementioned and other objects, a first aspect of the present invention provides an audio communication apparatus for an MRI apparatus that includes a first communication device provided at a side of a subject placed in a gantry provided in an examination room, and a second communication device provided at a side of an operation console provided outside the examination room. The second communication device and the first communication device are capable of unidirectional or bidirectional audio communication therebetween. Furthermore, an acoustic transducer for transmission and/or reception is provided in the first communication device, and is formed by a direct audio transmission type acoustic transducer that traits a sound to or from the subject by contacting the subject directly or via a protection member, without relying on an air as a transmission medium.

Thus, in the first aspect of the present invention, the acoustic transducer for transmission and/or reception provided on the subject side is formed by a direct audio transmission type acoustic transducer. Therefore, it becomes possible to clearly hear the sounds and voices transmitted from the operation console side to the subject side without relying on the great ability of the human auditory sense to exclude noise, even in a high-noise environment during operation of the MRI apparatus. Furthermore, it becomes possible to always clearly hear sounds and voices at the operation console side if the sounds and voices are transmitted from the subject side to the operation console side.

Still further, the audio communication apparatus of the first aspect of the present invention also functions as a hearing aid for a subject who has lost a hearing-related organ or the like, or a subject whose hearing function has reduced to a degree that is substantially equivalent to the loss of a hearing-related organ or the like. Thus, the practical usefulness of the apparatus is great.

According to a second aspect of the present invention based on the first aspect, the direct audio transmission type acoustic transducer may be formed from a nonmagnetic material.

Therefore, since the direct audio transmission type acoustic transducer is formed from a nonmagnetic material, it becomes possible to prevent the noise ounce in MRI images and the distortion and depletion of MRI image that are ascribable to direct audio transmission type acoustic transducers formed from a magnetic material such as iron or the like.

According to a third aspect of the present invention based on the second aspect, the direct audio transmission type acoustic transducer for transmission may include a piezoelectric bone conduction microphone, and the direct audio transmission type acoustic transducer for reception may include a piezoelectric bone conduction speaker.

Thus, the direct audio transmission type acoustic transducers are realized in specific fashions.

According to a fourth aspect of the present invention based on the third aspect, the first communication device may include a sound insulation earmuff that is wearable on the subject. Furthermore, the piezoelectric bone conduction microphone and/or the piezoelectric bone conduction speaker may be incorporated into the sound insulation earmuff so that an operation surface of the piezoelectric bone conduction microphone and/or the piezoelectric bone conduction speaker contacts the subject directly or via a protection member when the sound insulation earmuff is set on the subject

Therefore, this audio communication apparatus is easy for a subject to use. Furthermore, the apparatus makes it possible for the subject to more clearly hear the sounds and voices transmitted from the console side without the need to separately prepare earplugs or sound insulation earmuffs.

According to a fifth aspect of the present invention, an audio communication apparatus for an MRI apparatus includes a first communication device provided at a side of a subject placed in a gantry provided in an examination room, and a second communication device provided at a side of an operation console provided outside the examination room. The second communication device and the first communication device are capable of unidirectional or bidirectional audio communication therebetween. Furthermore, an acoustic transducer for reception is provided in the first communication device, and includes a bone conduction speaker. A music transmission device is provided which transmits a music signal from the side of the operation console, and which gives the music signal to the direct audio transmission type acoustic transducer so that music is reproduced and output.

Thus, in the fifth aspect of the present invention, the acoustic transducer for reception provided on the subject side is formed by a bone conduction speaker. Furthermore, the audio communication apparatus further includes the music transmission device which transmits a music signal from the side of the operation console, and which gives the music signal to the direct audio transmission type acoustic transducer so that music is reproduced and output. Therefore, it becomes possible to clearly hear the sounds and voices transmitted from the operation console side to the subject side without relying on the great ability of the human auditory sense to exclude noise, even in a high-noise environment during operation of the MRI apparatus. It also becomes possible to alleviate the mental burden of the subject before, during and after the MRI examination.

### BRIEF DESCRIPTION OF THE DRAWINGS

FIG. 1 is a schematic block diagram illustrating an embodiment of an audio communication apparatus for an MRI apparatus in accordance with the present invention;

FIG. 2 is a perspective view of a first embodiment of a subject-side transmitter-receiver shown in FIG. 1;

FIGS. 3A and 3B illustrate a state where the first embodiment of the subject-side transmitter-receiver shown in FIG. 1 is worn by a subject;

FIGS. 4A and 4B illustrate a state where a second embodiment of the subject-side transmitter-receiver shown in FIG. 1 is worn by a subject;

FIGS. 5A and 5B illustrate a state where a third embodiment of the subject-side transmitter-receiver shown in FIG. 1 is worn by a subject;

FIGS. 6A and 6B illustrate a state where a fourth embodiment of the subject- side transmitter-receiver shown in FIG. I is worn by a subject; and

FIG. 7 is a schematic sectional diagram illustrating a construction of a piezoelectric bone conduction speaker shown in FIGS. 2 to 6B and a piezoelectric bone conduction microphone shown in FIGS. 2 to 4B.

### DETAILED DESCRIPTION OF THE PREFERRED EMBODIMENTS

Preferred embodiments of the present invention will be described hereinafter with respect to the accompanying drawings. In the drawings, like reference characters represent like portions or components.

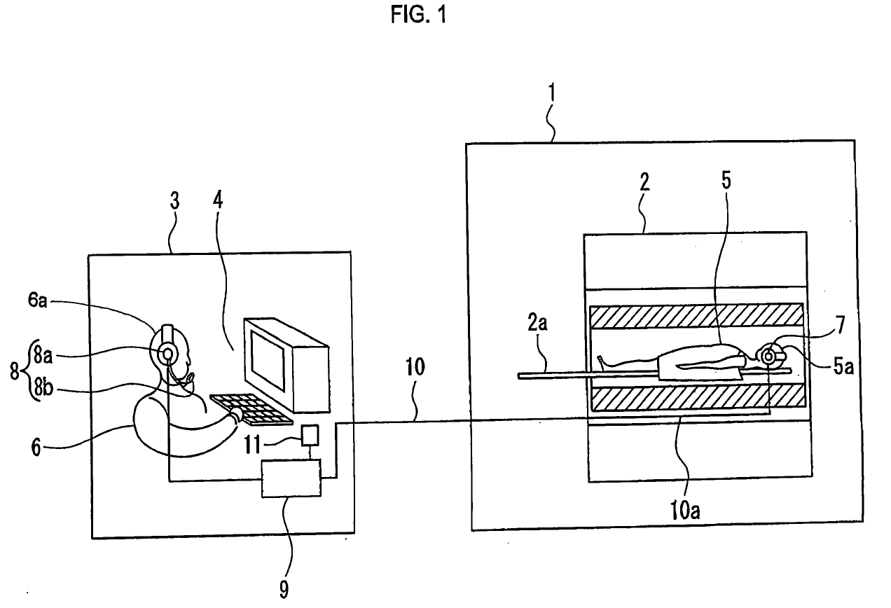

FIG. 1 is a schematic block diagram illustrating an embodiment of an audio communication apparatus for an MRI apparatus in accordance with the present invention

Referring to FIG. 1, a gantry (image taking system) 2, forming a main body of the MRI apparatus, is installed in an examination room 1 of the MRI apparatus. A bed 2a (described below) for transporting and holding a subject person is also installed in the gantry 2. A control room 3 for operating the apparatus is provided with an MRI apparatus-operating console 4 for positioning with respect to a target organ or region or an affected area of a subject (described below) and for acquiring necessary MRI images.

A subject 5 lies on the bed 2a in the gantry 2. An operator 6 sits at the console 4. Unidirectional or bidirectional audio communication is possible between the side of the subject 5 in the gantry 2 and the side of the console 4, specifically, between the subject 5, such as a patient or the like, and the operator 6, such as a doctor, a medical radiographer, a laboratory medical technologist, etc. In the apparatus shown in FIG. 1, bidirectional audio communication, that is, audio transmission and reception, can be performed between the subject 5 and the operator 6.

The audio transmission and reception is performed via transmission and reception-purpose acoustic transducers that convert sounds (vibrations) into electric signals or convert electric signals into sounds (vibrations). In this embodiment, the transmission and reception acoustic transducers provided at the side of the subject 5 are direct audio transmission type acoustic transducers.

The direct audio transmission type acoustic transducers transmit sounds (including voices) to the subject 5 or from the subject 5 not via air as a transmission medium, but by contacting a portion of the body of the subject 5, normally, a head portion 5a (including the face, ear portions; this applies in the below description as well) in a direct contact fashion or via a protection member.

The direct audio transmission type acoustic transducers used in the embodiment may be, for example, a bone conduction microphone or a laryngaphone for audio transmission, and a bone conduction speaker for audio reception.

In the apparatus shown in FIG. 1, a headset type transmitter-receiver employing a bone conduction microphone for audio transmission and a bone conduction speaker for audio reception is set as a subject-side transmitter-receiver 7, on the head portion 5a of the subject 5.

An operator-side transmitter-receiver 8 set on the head portion 6a of the operator 6 employs ordinary acoustic transducers that transmit voice and sound to or from the operator via air as a transmission medium. The operator-side transmitter-receiver 8 has a configuration in which a transmitter (microphone) 8b is connected to a headphone-type receiver 8a.

The two transmitter-receivers 7, 8 are interconnected via a bidirectional audio communication control device 9, and therefore are capable of transmission and reception with respect to each other. The audio communication control device 9 in this embodiment is designed so as to allow the switching between transmission and reception to be carried out through an operation at the side of the operator 6, and so as to prevent howling of the transmitter-receivers 7, 8.

Thus, in the audio communication apparatus for the MRI apparatus, the bone conduction microphone and the bone conduction speaker are used as the subject-side transmitter-receiver 7. Therefore, even in a high-noise environment during operation of the MRI apparatus, the subject 5 can clearly hear the voices and sounds transmitted from the side of the console 4 (operator 6) to the side of the subject 5, without relying on the highly-developed noise excluding ability of the human auditory sense. This advantage of the apparatus can still be enjoyed even if noise enter into an earhole of the subject 5. In this embodiment, earplugs or sound insulation earmuffs may be used as well. That is, the additional use thereof will allow even clearer hearing of voices and sounds from the side of the operator 6. The additional use of sound insulation earmuffs can easily be realized by assembling the bone conduction microphone and the bone conduction speaker of the subject-side transmitter-receiver 7 into a sound insulation earmuff set.

The above-described audio communication apparatus functions as a hearing aid for a subject who has lost a hearing-related organ or the like, or a subject whose hearing function has reduced to a degree that is substantially equivalent to the loss of a hearing-related organ or the like. Thus, the practical usefulness of the apparatus is great.

If component parts or the like of the subject-side transmitter-receiver 7, such as the bone conduction microphone and the bone conduction speaker as well as members for assembling the bone conduction microphone and speaker into the headset transmitter-receiver, etc., are formed of nonmagnetic materials, it becomes possible to acquire MRI images with reduced magnetic effects (noise, depletion and distortion in image). As for a communication line 10 from the audio communication control device 9 to the subject-side transmitter-receiver 7, if a nonmagnetic material wire, for example, a copper wire, is used for a portion of the communication line 10 that has a magnetic effect on MRI images, for example, a communication line portion 10a located within the examination room 1 and, particularly, wit the gantry 2, the magnetic effects on MRI images (noise, depletion, distortion in image) can also be reduced.

In the audio communication apparatus for the MRI apparatus shown in FIG. 1, it is also possible to provide a music transmission device 11 that transmits desired music signals from the side of the console 4 at a desired time, to a direct audio transmission type acoustic transducer, for example, a bone conduction speaker, of the subject-side transmitter-receiver 7 so that music is reproduced from the music signals, and is output.

This arrangement achieves the advantage of alleviating the mental burden of the subject 5 (discomfort, anxiety and, particularly, loneliness) by presenting music to the subject 5 at a desired time, particularly, during the MRI operation, in addition to the aforementioned advantage of clear audibility of voices and sounds transmitted from the console 4 side to the subject 5 side. Furthermore, it becomes possible to flexibly cope with a sudden change in the condition of the subject 5 or an emergency sign from the subject 5.

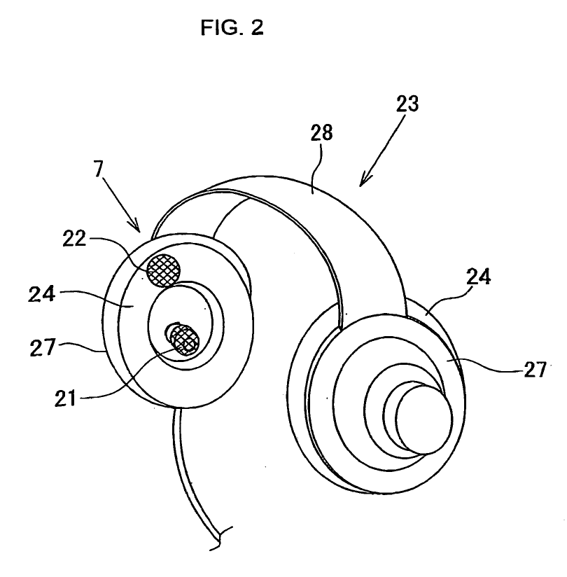

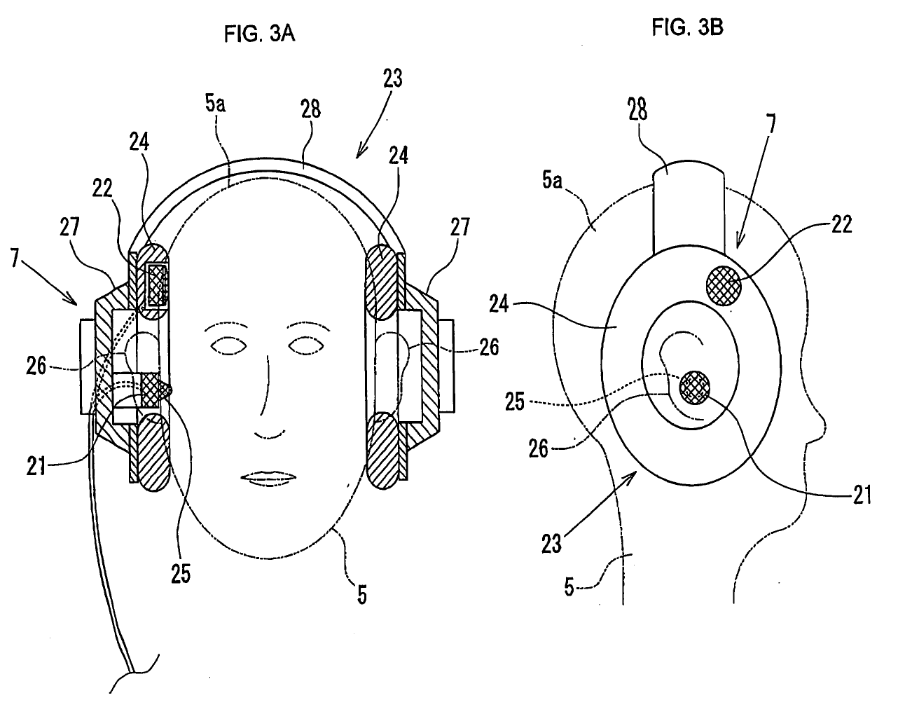

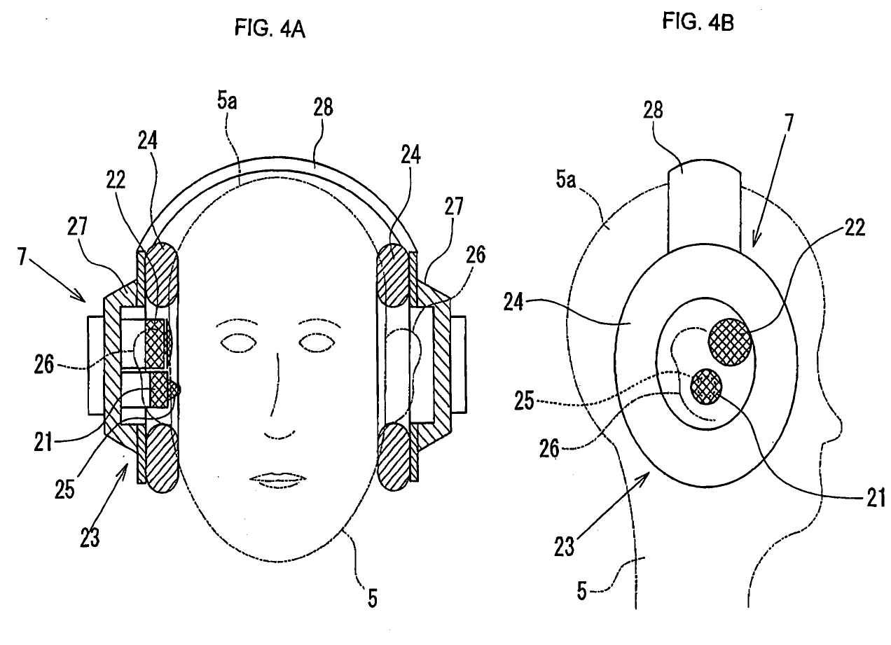

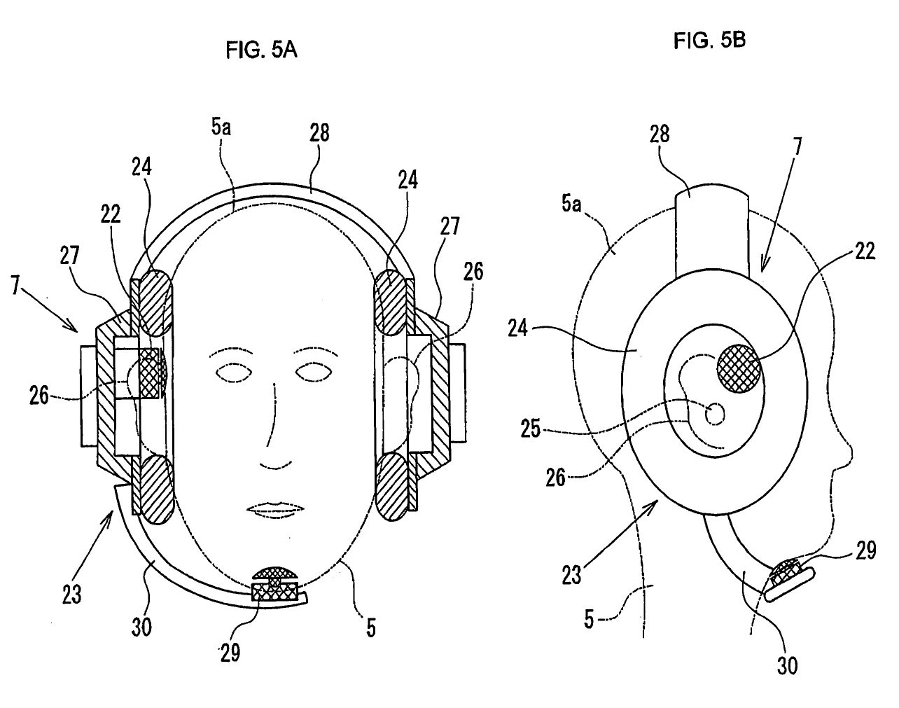

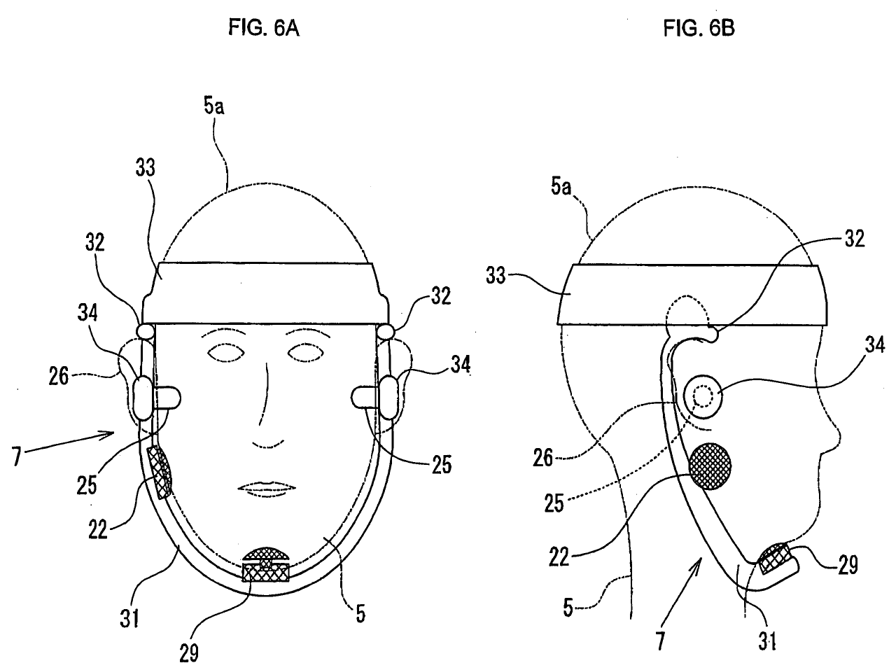

Embodiments of the subject-side transmitter-receiver 7 will be next described with respect to FIGS. 2 to 6. FIGS. 2, 3A and 3B show a first embodiment of the subject-side transmitter-receiver 7 shown in FIG. 1. FIGS. 4A and 4B show a second embodiment of the subject-side transmitter-receiver 7. FIGS. 5A and 5B show a third embodiment of the subject-side transmitter-receiver 7. FIGS. 6A and 6D show a fourth embodiment of the subject-side transmitter-receiver 7.

FIGS. 3A, 4A and SA each show a fragmentary sectional view of the subject-side transmitter-receiver 7 set on a subject, taken along a plain that faces the subject front side. FIGS. 3B, 4B and 5B each show portions of the subject-side tansmitter-receiver 7 in a view taken from the left side in FIGS. 3A, 4A and 5A (right side of the subject). FIG. 6A shows the subject-side transmitter-receiver 7 set on the subject, in a view taken from the subject front side, and FIG. 6B shows a view thereof taken from the left side in FIG. 6A (right side of the subject).

FIGS. 2 to 3B (first embodiment) and FIGS. 4A and 4B (second embodiment) show subject-side transmitter-receivers 7 in which a bone conduction microphone 21 formed by a nonmagnetic piezoelectric element (hereinafter, referred to as “piezoelectric bone conduction microphone”) is used for transmission and a bone conduction speaker 22 formed by a nonmagnetic piezoelectric element (hereinafter, referred to as “piezoelectric bone conduction speaker”) is used for reception.

In either embodiment, the piezoelectric bone conduction microphone 21 and the piezoelectric bone conduction speaker 22 are integrated with a headset-type sound insulation earmuff set 23. Although the sound insulation earmuff set 23 has two ear pads 24, for the sake of explanatory simplicity, the following description will be given with respect to only one of the ear pads 24. The piezoelectric bone conduction microphone 21 is positioned in a substantially central portion of an interior of an ear pad 24 of the sound insulation earmuff set 23 (the central portion corresponds to the normal position of an earhole 25 of an ordinary subject 5), and is designed so that a distal end portion of the piezoelectric bone conduction microphone 21 will enter the earhole 25 of the subject 5 when the subject 5 puts the sound insulation earmuff set 23 on. The piezoelectric bone conduction speaker 22 is housed within the ear pad 24 (at a location corresponding to the surrounding of a pinna 26 of an ordinary subject 5) in the embodiments shown in FIGS. 2, 3A and 3B. The position of the piezoelectric bone conduction speaker 22 is arbitrary as long as it is within the ear pad 24. In the embodiments of FIGS. 2, 3A and 3B, the piezoelectric bone conduction speaker 22 is disposed at a position that is in an upper region of the ear pad 24 and slightly forward of a front-rear-direction center thereof.

In the embodiment of FIGS. 4A and 4B, the piezoelectric bone conduction speaker 22 is disposed at a position that is inward and is relatively forward or relatively diagonally upwardly forward of a central portion of the ear pad 24 (corresponding to the location of the earhole 25 of an ordinary subject 5).

In FIGS. 2 to 4B, the piezoelectric bone conduction microphone 21 and the piezoelectric bone conduction speaker 22 are incorporated so that when the sound insulation earmuff set 23 is set on the subject 5, the operating surface of each one of the piezoelectric bone conduction microphone 21 and the piezoelectric bone conduction speaker 22 contacts the subject 5 (the head portion 5a including the earhole 25) in a direct contact fashion or via a protection member (not shown) that is formed by a protection cover, a cushion member, etc. The ear pad contact pressure can be set by suitably selecting a material of a headband 28 that connects right and left-side earmuff housings 27 of the sound insulation earmuff set 23, and can be adjusted through adjustment of the right-left length of the headband 28 (right-left direction in FIGS. 3A and 4A).

The ear pads 24 are provided on the inner surfaces of the right and left-side housings 27, and have the same configuration (dimensions) and are formed from the same material. However, the piezoelectric bone conduction microphone 21 and the piezoelectric bone conduction speaker 22 are provided in one of the right and left-side housings 27 combined with the ear pads 24, that is, in the right-side housing 27 combined with the ear pad 24 in the embodiments. The microphone 21 and the speaker 22 are connected to and held by the housing 27.

Ordinary type sound insulation ear muff sets are provided with functions and structures that facilitate the setting on and removal from a subject's head portion 5a, and that provides an appropriate fitting feel to the subject head portion 5a. Similar functions and structures are provided (not shown) in the sound insulation earmuff sets 23 of the embodiments as well.

In the embodiments shown in FIGS. 2 to 4B, the piezoelectric bone conduction microphone 21 may be disposed at a position that is relatively inward of the ear pad 24 of the sound insulation earmuff set 23 and is relatively low (the position is directly below the site corresponding to the earhole 25 of an ordinary subject 5). The piezoelectric bone conduction microphone 21 may also be provided within the ear pad 24 of the sound insulation earmuff set 23 (in the case of application to the embodiment shown in FIGS. 3A and 3B, the piezoelectric bone conduction microphone 21 is provided at a position opposite from the piezoelectric bone conduction speaker 22 in FIG. 3B).

FIGS. 5A to 6B show subject-side transmitter-receivers 7 in which a laryngaphone formed by a nonmagnetic piezoelectric element (hereinafter, referred to as “piezoelectric laryngaphone”) 29 is used as a direct audio transmission type acoustic transducer. Thus, it is possible to employ the piezoelectric laryngaphone 29 instead of the piezoelectric bone conduction microphone.

In the embodiment shown in FIGS. 5A and 5B, the piezoelectric bone conduction speaker 22 is disposed within the ear pad 24 of the headset type sound insulation earmuff set 23 as in the embodiment shown in FIGS. 4A and 4B. The piezoelectric laryngaphone 29 is connected to and supported by one of the right and left-side housings 27 of the sound insulation earmuff set 23, that is, the right-side housing 27 in the embodiment shown in FIGS. 5A and 5B, via an arm 30 extending therefrom to a position corresponding to the throat of the subject 5.

In the embodiment shown in FIGS. 6A and 6B, the piezoelectric bone conduction speaker 22 is provided so as to contact a slightly lower portion of the pinna 26 of an ordinary subject 5. The piezoelectric bone conduction speaker 22 and the piezoelectric laryngaphone 29 are connected to and supported by a generally U-shaped arm 31 that extends from a site corresponding to one of the ears of the subject 5 to a site corresponding to the other ear via a throat portion. Arc-shaped ear hooks 32 are formed at right and left-side ends of the arm 31 so that the arm 31 can be hooked on the ears of the subject 5. An annular band 33 is a retainer for preventing the arm 31 and therefore the piezoelectric bone conduction suer 22 and the piezoelectric laryngaphone 29 from shifting from the position indicated in FIGS. 6A and 6B or falling off. The baud 33 is formed of an expandable band-shaped member.

In FIGS. 5A and 6B, the piezoelectric laryngaphone 29 and the piezoelectric bone conduction speaker 22 are incorporated into the sound insulation earmuff set 23, the arm 30, or the arm 31 so that when the sound insulation earmuff set 23, or the ear hooks 32 and the band 33 are set on the subject 5, the operating surface of each one of the laryngaphone 29 and the speaker 22 contacts the subject 5 (throat portion or head portion) in a direct contact fashion or via a protection member (not shown) that is formed by a protection cover, a cushion member, etc.

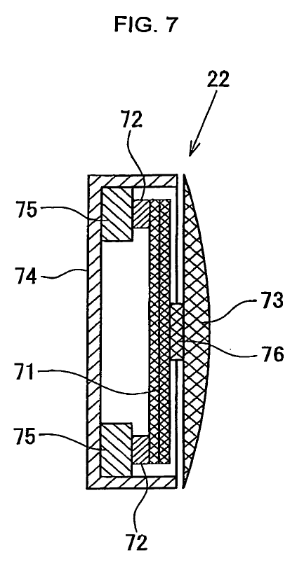

FIG. 7 is a schematic sectional diagram illustrating a construction of the piezoelectric bone conduction per 22 shown in FIGS. 2 to 6B and the piezoelectric bone conduction microphone 21 shown in FIGS. 2 to 4B.

As shown in FIG. 7, the piezoelectric bone conduction speaker 22 and the piezoelectric bone conduction microphone 21 are constructed in the following manner. That is, a piezoelectric bimorph (ceramic) element 71 is retained by an elastomer member 72. The piezoelectric bimorph element 71 is caused to vibrate a vibrator 73 (i.e., output voices and sounds) in accordance with audio signals, or is caused to output audio signals (electric signals) by vibrations of the vibrator 73 caused by voices and sounds (vibrations) The elastomer member 72 is supported on a support member 75 that is fixed to an inner surface of a case 74. The piezoelectric bimorph element 71 is housed in the case 74. The vibrator 73 is constructed so that at least a surface (operation surface) 76 thereof is positioned outside the case 74.

It is to be noted herein that the construction of the piezoelectric bone conduction speaker 22 and the piezoelectric bone conduction microphone 21 is not restricted by the construction exemplified in FIG. 7.

According to the embodiments shown in FIGS. 2 to 5B, since the sound insulation earmuff set 23 is provided, improved facility for the subject 5 is achieved. Furthermore, it becomes possible for the subject 5 to hear the voices and sounds transmitted from the console side with improved clarity, without the need to separately prepare earplugs or a sound insulation earmuff sew

According to the embodiments shown in FIGS. 2 to 6B, it is possible to prevent the noise occurrence in MRI images and the distortion and depletion of MRI images ascribable to direct audio transmission type acoustic transducers formed of a magnetic material such as iron or the like. If all the component members of the subject-side transmitter-receiver 7, such as the sound insulation earmuff set 23, the annular band 33, the arm 30, the arm 31, etc., are formed of non-magnetic materials, the advantage of preventing the noise occurrence in MRI images and the distortion and depletion of MRI images will further increase.

In the embodiment shown in FIG. 6, earplugs 34 may be used in accordance with the need since this audio communication apparatus does not have a sound insulation earmuff set. The use of the earplugs 34 will make it possible to more clearly hear voices and sounds transmitted from the console side.

In the embodiments shown in FIGS. 2 to 4B, the audio communication apparatus may be constructed so that each of the right and left sides of the head portion 5a of the subject 5 is provided with one or more piezoelectric bone conduction microphones 21 and one or more piezoelectric bone conduction speakers 22. Likewise, the audio communication apparatuses shown in FIGS. 5A to 6B may be constructed so that each of the right and left sides of the head portion 5a of the subject 5 is provided with one or more piezoelectric bone conduction speakers 22. If, in any one of the apparatuses shown in FIG. 2 to 6B, piezoelectric bone conduction speakers 22 are provided on the right and left sides of the head portion 5a of the subject 5, it becomes possible to realize stereophonic reproduction of stereo music signals received from the music transmission device 11 shown in FIG. 1, so that the subject 5 can listen to stereo sounds of music. Thus, it becomes possible to further reduce the subject's discomfort, anxiety and, particularly, loneliness.

Furthermore, if the microphone 21 and the speaker 22 are disposed close to each other as in the embodiments shown in FIGS. 3A to 4B, it is desirable to provide an arrangement in which the microphone 21 and the speaker 22 can be operated selectively in a switching fashion so as to prevent howling in conjunction with the bidirectional communication with the operator side.

1. An audio communication apparatus for an MRI apparatus, comprising:
a first communication device provided at a side of a subject placed in a gantry provided in an examination room;
a second communication device provided at a side of an operation console provided outside the examination room, the second communication device and the first communication device being capable of unidirectional or bidirectional audio communication therebetween; and
an acoustic transducer which is provided in the first communication device, and which can perform at least one of transmission and reception, the acoustic transducer being formed by a direct audio transmission type acoustic transducer that transmits a sound to or from the subject by contacting the subject directly or via a protection member, without relying on an air as a transmission medium.

2. The audio communication apparatus according to claim 1, wherein the direct audio transmission type acoustic transducer is formed from a nonmagnetic material.

3. The audio communication apparatus according to claim 2, wherein the direct audio transmission type acoustic transducer for transmission includes a piezoelectric bone conduction microphone, and the direct audio transmission type acoustic transducer for reception includes a piezoelectric bone conduction speaker.

4. The audio communication apparatus according to claim 3,
wherein the first communication device comprises a sound insulation earmuff that is wearable on the subject, and
wherein the piezoelectric bone conduction microphone and/or the piezoelectric bone conduction speaker is incorporated into the sound insulation earmuff so that an operation surface of the piezoelectric bone conduction microphone and/or the piezoelectric bone conduction speaker contacts the subject directly or via a protection member when the sound insulation earmuff is set on the subject.

5. An audio communication apparatus for an MRI apparatus, comprising:
a first communication device provided at a side of a subject placed in a gantry provided in an examination room;
a second communication device provided at a side of an operation console provided outside the examination room, the second communication device and the first communication device being capable of unidirectional or bidirectional audio communication therebetween;
an acoustic transducer for reception which is provided in the first communication device, the acoustic transducer for reception including a bone conduction speaker, and
a music transmission device that transmits a music signal from the side of the operation console, and gives the music signal to the direct audio transmission type acoustic transducer so that music is reproduced and output.
